SEQUENCE LISTING (7 sequences)

SEQ ID NO: 1 - skipped

SEQ ID NO: 2 - RNA, 10 nt, synthetic construct
  source 1..10: mol_type = other RNA
atcgtgcaac

SEQ ID NO: 3 - RNA, 17 nt, synthetic construct
  source 1..17: mol_type = other RNA
atcgtgcaacgtagcat

SEQ ID NO: 4 - RNA, 30 nt, synthetic construct
  source 1..30: mol_type = other RNA
atcgtgcaacgtagcataataagtgatcgt

SEQ ID NO: 5 - RNA, 17 nt, synthetic construct
  source 1..17: mol_type = other RNA
atcgtgcaacgtagcat

SEQ ID NO: 6 - DNA, 15 bp, synthetic construct
  source 1..15: mol_type = other DNA
gatcaacggtacgac

SEQ ID NO: 7 - DNA, 17 bp, synthetic construct
  source 1..17: mol_type = other DNA
atcgtgcaacgtagcat

(19) United States
(12) Patent Application Publication
(10) Pub. No.: US 2024/0418721 A1
(43) Pub. Date: Dec. 19, 2024

(54) Methods and Kits for Ribonuclease Detection

(71) Applicants: Lance Philip Ford, Austin, TX (US); Joseph Francis Krebs, Austin, TX (US)

(72) Inventors: Lance Philip Ford, Austin, TX (US); Joseph Francis Krebs, Austin, TX (US)

(21) Appl. No.: 18/742,862

(22) Filed: Jun. 13, 2024

Related U.S. Application Data
(60) Provisional application No. 63/508,309, filed on Jun. 15, 2023.

(51) Int. Cl. G01N 33/573; G01N 33/543

(52) U.S. Cl. CPC G01N 33/573; G01N 33/54346; G01N 33/54388; G01N 2333/922

(57) ABSTRACT

A method for detecting ribonucleases uses a reagent fluid containing ribonucleic acid (RNA) tagged with two different affinity tags which is suspended in a buffered solution with a block copolymer surfactant. A plurality of detectable nanoparticles are coated with a receptor that can bind to one of the two affinity tags but not both. A test strip featuring a test line with receptors capable of binding one of the two affinity tags is provided. During the test, a sample containing ribonuclease is mixed with the reagent fluid and the running buffer. As the mixture travels along the strip, it reaches the test line where the nanoparticles are expected to bind. However, ribonuclease reduces this binding, therefore indicating the presence of the enzyme in the sample. This method provides a visual indication of ribonuclease activity based on the interaction and binding patterns of the nanoparticles on the test strip.

### CROSS-REFERENCE DATA

This patent application claims a priority date benefit from a co-pending U.S. Provisional Patent Application No. 63/508,309 filed Jun. 15, 2023 and entitled “Rapid test to detect ribonuclease,” which is incorporated herein by reference in its entirety.

### SEQUENCE LISTING XML

A Sequence Listing XML file entitled “RNAase patent sequences” has been created and submitted electronically via the USPTO's EFS-Web system on 29 Aug. 2024. The size of the file is 6.33 KB. The content of the Sequence Listing is incorporated herein by reference in its entirety. The Sequence Listing contains the nucleotide sequences necessary to understand the invention and is provided in both the application as-filed and in the accompanying XML file.

### BACKGROUND

Without limiting the scope of the invention, its background is described in connection with methods and kits for ribonuclease detection. More particularly, the invention describes methods for detecting ribonuclease presence, such as in liquids and on hard surfaces, using an RNA attached to two affinity tags in a buffered aqueous solution containing a block copolymer surfactant.

Ribonucleic acids (RNA) play a central role in many cellular and biological processes including protein synthesis, gene regulation, cellular differentiation, cellular immunity, apoptosis, and cancer pathogenesis. Scientists produce and study cellular RNAs to better understand these processes. RNAs are powerful diagnostic markers for gene expression and the detection of human diseases. RNAs can now be manufactured at a large scale to produce vaccines, therapeutic agents, and diagnostic reagents. However, cellular and synthetic RNA molecules are very susceptible to degradation during synthesis, processing, purification, and storage.

Ribonucleases (RNases) are a class of enzymes crucial for the metabolism and processing of RNA molecules. They function by catalyzing the cleavage of RNA, hydrolyzing the phosphodiester bonds between nucleotides. This enzymatic activity is essential in various biological processes, including the degradation of RNA molecules that are no longer needed or are faulty, as well as in the maturation and processing of functional RNAs such as messenger RNA (mRNA), transfer RNA (tRNA), and ribosomal RNA (rRNA).

There are several types of ribonucleases, each specialized for different roles within the cell. For example, RNase III plays a significant role in the processing of precursor rRNA into mature rRNA, essential for ribosome assembly. RNase H is involved in removing RNA primers used during DNA replication and is vital for the synthesis of the DNA strand. Another important ribonuclease, RNase P, is involved in generating mature tRNA molecules by cleaving their precursor forms.

Outside of cellular metabolism, ribonucleases have practical applications in biotechnology and medicine. They are used in molecular biology laboratories to remove RNA contamination from DNA preparations and to study RNA sequences and structures. In medical research, engineered ribonucleases are explored for their potential as therapeutic agents. For instance, some ribonucleases possess antiviral and anticancer properties, making them candidates for drug development. They target RNA molecules within pathogens or cancer cells, leading to their degradation and inhibiting their ability to proliferate or infect.

Ribonucleases (RNases) play a number of important roles in living organisms including RNA precursor processing, RNA maturation, clearance of unwanted RNA, immune response, defense against viruses, angiogenesis, and downregulation of gene expression. Ribonucleases can be used as biomarkers for the diagnoses of a variety of diseases, including pancreatic and prostate cancers, and can be used as therapeutic agents to fight cancer and chronic viral infections. RNases can also be used to remove unwanted RNA from DNA and protein products during bioprocessing. While RNases have beneficial uses as biomarkers, therapeutic agents, and bioprocessing reagents, they can cause the undesirable breakdown of valuable RNA molecules produced for important research, diagnostic, and therapeutic applications. Great care must be taken to detect and control unwanted ribonucleases in manufacturing and research environments used to produce and analyze RNA.

Over the years a number of assays have been developed to detect active ribonuclease enzymes in biological samples. Examples of these detection methods include zymograms, radionuclide-labeled RNA electrophoresis assays, and spectrophotometric assays. Fluorescence assays using spectrofluorometers and plate readers have become particularly popular in recent years. For example, Keleman (Kelemen B R, Klink T A, Behlke M A, Eubanks S R, Leland P A, Raines R T. Nucleic Acids Res. 1999; 27 (18): 3696-3701. doi:10.1093/nar/27.18.3696.) and James (James D A, Woolley G A. Anal Biochem. 1998; 264 (1): 26-33. doi:10.1006/abio.1998.2824.) describe the use of a spectrofluorometer to monitor the cleavage of the quenched fluorescently-labeled RNA oligonucleotide substrate by ribonuclease. Trubetskoy (Trubetskoy V S, Hagstrom J E, Budker V G. Anal Biochem. 2002; 300 (1): 22-26. doi:10.1006/abio.2001.5442.) describes the use of a spectrofluorometer to monitor the cleavage of self-quenched RNA substrates to measure RNase activity. Since the bulk fluorescence signal is arbitrary, these tests generally require both a negative and positive control to be performed with sample analysis to provide a requisite comparison.

U.S. Pat. No. 6,773,885 describes the compositions and uses of RNA oligonucleotides labeled with a fluorescent dye and a quenching group to detect ribonucleases using either a spectrofluorometer or by visible inspection (without a spectrofluorometer). Cleavage of the RNA by ribonuclease in bulk solution releases the fluorescent reporter dye from the quenching group, producing a visually detectable fluorescent signal upon irradiation with ultraviolet light. The visual test must be performed in a dark environment using “short wave” UV irradiation for the highest sensitivity ribonuclease detection.

Modern techniques for detecting ribonucleases are integral to various scientific and medical fields but have several drawbacks. These methods can lack specificity, potentially leading to false-positive results due to interfering substances, and may not be sensitive enough to detect low ribonuclease levels crucial for some experiments. Procedures like zymography require considerable time and expertise, complicating their use in less specialized settings. Additionally, many detection techniques demand highly purified samples to avoid inaccuracies caused by contaminants, increasing preparation costs and complexity. Advanced detection technologies like fluorescence-based assays or mass spectrometry, while effective, are often prohibitively expensive for smaller labs. Furthermore, ribonuclease activity assays can be sensitive to environmental conditions like pH and temperature, which, if uncontrolled, can lead to inconsistent results. Lastly, many methods do not provide quantitative information about RNase concentration or activity, limiting their utility in detailed biological or clinical analysis.

It would be highly useful to create a portable, sensitive test for ribonuclease detection in ambient light conditions that does not require a spectrofluorometer or irradiation with UV light.

### SUMMARY

Accordingly, it is an object of the present invention to overcome these and other drawbacks of the prior art by providing a novel method for detecting ribonuclease that improves on the limitations of the prior art.

It is another object of the present invention to provide a novel method for detecting ribonuclease with improved sensitivity and portability.

It is a further object of the present invention to provide a novel method for detecting ribonuclease that may be completed in minutes and does not require complex laboratory equipment.

It is yet another object of the present invention to provide a novel method and a kit for detecting ribonuclease on a hard surface.

It is yet a further object of the present invention to provide a novel method for detecting ribonuclease that operates without the need to use ultraviolet light.

The novel method for detecting ribonuclease uses a multi-component system. First, a reagent fluid is prepared containing ribonucleic acid (RNA) that is tagged with two different affinity tags and is suspended in a buffered solution with a block copolymer surfactant. Next, a plurality of detectable nanoparticles are coated on their surface with a receptor that can bind to one of the two affinity tags but not both. A test strip is also prepared, featuring a test line with receptors capable of binding one of the two affinity tags. To perform the test, an aqueous sample suspected of containing ribonuclease may be mixed with the reagent fluid. This mixture is then combined with a running buffer to enable the flow of the detectable nanoparticles along the test strip. As this mixture travels along the strip, it reaches the test line. Typically, detectable nanoparticles would bind to the test line. However, the presence of ribonuclease in the sample reduces the binding of the nanoparticles to the test line, thereby indicating the presence of the enzyme in the sample. This method provides a visual or optically measurable indication of ribonuclease activity based on the interaction and binding patterns of the nanoparticles on the test strip.

The test strip may feature both a test line and a control line. The test line functions as described above, detecting the presence of ribonucleases based on a decrease in nanoparticle binding. The control line may contain receptors that bind to the nanoparticles regardless of the presence of ribonucleases, providing a consistent signal to confirm the test's validity. This binding at the control line can occur directly or through a linker compound that serves an as intermediary connection point for both the nanoparticles and the control line's receptors. The method employs a reagent fluid in which RNA is suspended in a nonionic block copolymer surfactant solution. This surfactant, specifically a Pluronic F68, consists of a central hydrophobic polypropylene oxide chain flanked by hydrophilic polyethylene oxide chains, enhancing the mixture's stability and interaction dynamics. Additionally, the test strip used is typically made from nitrocellulose, a material chosen for its effective binding and fluid-wicking properties. This setup ensures a robust and reliable system for ribonuclease detection, with built-in controls to verify test functionality and accuracy.

Also described is a method for detecting ribonuclease on a hard surface. It involves several precise steps. A nuclease-free swab may be saturated with a reagent fluid, which contains ribonucleic acid labeled with a first affinity tag and a second affinity tag dissolved in a buffered solution that includes a block copolymer surfactant. The swab is used to wipe the test surface, ensuring any ribonuclease present is collected. After swiping, the swab is submerged in the remaining reagent fluid. Detectable nanoparticles, coated with receptors that can bind to either of the two affinity tags but not both, are then introduced along with a running buffer. This mixture is applied to a test strip equipped with a test line that also contains receptors capable of binding to one of the affinity tags. As the mixture flows along the test strip, the presence of ribonuclease on the original surface is indicated by reduced binding of nanoparticles at the test line, demonstrating whether ribonuclease contamination of the surface exists.

Finally, a test kit for detecting ribonucleases is described and includes several components designed to work together for effective ribonuclease detection. It contains a reagent fluid where ribonucleic acid is tagged at each end with distinct affinity tags and suspended in a buffered solution enriched with a block copolymer surfactant. Also included are detectable nanoparticles coated with receptors that specifically bind to one of the two affinity tags, but not both. The kit also features a test strip, which is equipped with a test line that has surface-immobilized receptors capable of binding one of the affinity tags on the RNA. To assist in facilitating the flow of these nanoparticles along the test strip, a running buffer is provided. This setup allows for the efficient and targeted detection of ribonucleases based on their interaction with the tagged RNA and the subsequent binding events on the test strip.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. This patent application contains at least one drawing executed in color. Understanding that these drawings depict only several embodiments in accordance with the disclosure and are, therefore, not to be considered limiting of its scope, the disclosure will be described with additional specificity and detail through use of the accompanying drawings, in which:

FIG. 1 shows the tagged oligonucleotide-dependent visualization of the test line using coated gold nanoparticles, as it pertains to Example 1.

FIG. 2 is a comparison view of two test strips demonstrating the reliable performance of a control line as it pertains to Example 2.

FIG. 3 shows the test performance of Example 3 in a dose-dependent manner.

FIG. 4 illustrates test strips after testing samples without pre-incubation.

FIG. 5 illustrates the same but after a preincubation period.

FIG. 6 illustrates a comparison of test strips with various concentrations of Pluronic F68.

FIG. 7 illustrates test strip results using quantum dot detectable nanoparticles pertaining to Example 7.

FIG. 8 illustrates optimized test and control results pertaining to Example 8.

FIG. 9 shows the detection of ribonuclease on a hard surface, pertaining to Example 9.

FIG. 10 shows various sequences described in the specification.

### DETAILED DESCRIPTION OF PREFERRED EMBODIMENTS OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

The method of the invention teaches a sequence of steps to detect the presence or absence of ribonuclease. It may be adapted to be performed as a traditional lateral flow test. Generally speaking, a lateral flow test is a diagnostic device used to confirm the presence or absence of a target analyte in a sample. It operates on the principles of capillary action to transport the sample across the different components of the test strip. Typically, the test strip consists of several overlapping layers, including a sample pad, a conjugate pad, a reactive test line, and a control line, all mounted on a backing card, such as a nitrocellulose card.

The process usually begins when a fluid sample is added to a vial containing a reagent fluid. The mixture including the reagent fluid and the running buffer may then be applied to the sample pad. Alternatively, a test strip may be submerged into a vial containing the mixture of the test sample, running buffer, and the reagent fluid. Conventional pads often contain buffers that prepare the sample by maintaining the pH and salinity conducive to the test. The sample then flows to the conjugate pad, which contains pre-coated antibodies or antigens tagged with detectable nanoparticles. As the sample mixture continues to move due to capillary action, it reaches the test line area, where specific antibodies or antigens are immobilized. The intact RNA reagent enables the detectable nanoparticles to bind the immobilized antibodies or antigens, capturing the detectable nanoparticles and forming a visible line. If the target analyte is present in the sample, it cleaves the RNA, which prevents it from binding the detectable nanoparticles to the test line. The intensity of the test line can sometimes be inversely proportional to the amount of the target analyte present in the sample.

Beyond the test line is the control line, which is crucial for validating the test's functionality. Regardless of the presence of the target analyte, this line captures excess nanoparticles, confirming that the sample has flowed through the strip properly and that the reagents are working as expected. Finally, the absorbent pad at the end of the strip (wick) acts as a sink to draw the fluid through the test strip and to ensure consistent flow.

For the purposes of the present invention, components, and fluids used in the test are significantly changed from what is known in the prior art. More specifically, a novel reagent fluid may be provided. The reagent fluid may include a ribonucleic acid, in particular having at least 5 or more nucleotides. In embodiments, the ribonucleic acid of the reagent fluid may include between 5 and 30 nucleotides, such as at least 5 nucleotides, at least 10 nucleotides, at least 15 nucleotides, at least 20 nucleotides, at least 25 nucleotides or as much as 30 nucleotides.

The length and sequence composition of the ribonucleic acid described herein may be optimal for the detection of RNase A. Although ribonucleic acids as short as six nucleotides can be used to detect RNase A, higher sensitivity detection be achieved using ribonucleic acids with lengths between 17 and 30 nucleotides (see Example 5 below). The ability of the ribonucleic acid substrate to be cleaved by a particular RNase can be enhanced by modifying the sequence of the oligonucleotide. In some embodiments, the oligonucleotide contains C and U nucleotides to permit cleavage by RNase A, RNase B, and RNase C. If the oligonucleotide sequence contains each of the four nucleotides, A, C, G, and U, it can be cleaved by a number of different RNase molecules. The invention, therefore, can be used to detect other ribonuclease enzymes including RNase T1, RNase I, mung bean nuclease, and micrococcal nuclease.

The ribonucleic acid may be labeled with two different affinity tags. In embodiments, the ribonucleic acid may be attached to the first affinity tag on the first end thereof and also attached to the second affinity tag on the second end thereof. The first end and the second end of the ribonucleic acid attached to the affinity tags may be separated from each other, such as by at least 6 nucleotides. In one example, the affinity tag may be attached on the 5′ end and the second affinity tag may be attached at the 3′ end of the ribonucleic acid.

These affinity tags permit simultaneous binding of the ribonucleic acid to two specific receptors. A variety of suitable affinity tags may be used for the purposes of the present invention. For example, the first and/or the second affinity tag may include a biotin, a peptide, a carbohydrate, a steroid, a hormone, a dye molecule, another small molecule epitope, or combinations thereof. Advantageously, because the affinity tags bind to receptors in the reagent fluid, fluorescence-quenching tags are not required for the oligonucleotides.

A variety of receptors may be used for the purposes of the invention, such as a protein (such as streptavidin), an antibody, an extracellular receptor, a metal-binding protein, an enzyme, an aptamer, or an immobilized nucleic acid polymer.

A plurality of detectable nanoparticles may also be provided as part of the method and the kit of the present invention. In particular, the plurality of detectable nanoparticles may have their surfaces coated with a receptor capable of binding either the first affinity tag or the second affinity tag but not both. Detectable nanoparticles can be selected from the group consisting of: colloidal gold nanoparticles, quantum dots nanoparticles, lanthanide beads nanoparticles, R-phycoerythrin nanoparticles, polystyrene nanoparticles, allophycocyanin (APC) nanoparticles, and latex bead nanoparticles.

Furthermore, while in some embodiments, the affinity tag may be directly coated on the nanoparticle itself (referred to here as a direct coating), in other embodiments, the affinity tag binds the oligonucleotide to a receptor previously coated on the nanoparticles (referred to here as an indirect coating). The other affinity tag may bind to a receptor immobilized at the test line as the nanoparticles flow through the strip by capillary action.

In embodiments, detectable nanoparticles may be coated with a receptor that binds one of the first or the second affinity tags on the ribonucleic acid. In one embodiment, the affinity tag is a biotin and the receptor is a biotin-binding protein. The detectable nanoparticles can be passively coated with the receptor using electrostatic and hydrophobic interactions. In other embodiments, they can be chemically attached onto functionalized nanoparticles using a crosslinking agent, such as 1-Ethyl-3-(3′-dimethylaminopropyl) carbodiimide (carbodiimide). Other attachment chemistry methods can be also used, as the invention is not limited in this regard. In embodiments where the test line is provided together with an adjacent control line, the receptor may directly bind to the control line, or bind an affinity group attached to a linker compound that can also bind to the control line. In one exemplary embodiment, streptavidin may be attached to the surface of carboxyl-modified colloidal gold nanoparticles using carbodiimide. In another example, streptavidin may be attached to the surface of carboxyl quantum dots using carbodiimide. In further examples, the plurality of detectable nanoparticles may be coated avidin or modified avidin.

In some embodiments, the ribonucleic acid substrate can be precoated onto the plurality of detectable nanoparticles before encountering the test sample. In other embodiments, the ribonucleic acid substrate can be coated onto the nanoparticles after mixing with the test sample. Furthermore, the sample-oligonucleotide mixture can be either tested right away or incubated for a predetermined duration of time. In some embodiments, this incubation duration of time may be at least 1 minute, at least 2 minutes, at least 3 minutes, at least 4 minutes, at least 5 minutes, at least 10 minutes, at least 15 minutes, at least 20 minutes, at least 25 minutes, or up to 30 minutes. Longer incubation time may help to increase the sensitivity of the test. The test sample may be provided at a room temperature or generally within the temperature range of 20-37° C.

A test strip may be provided for the purposes of the present invention. The test strip may include at least one test line configured to bind to ribonucleic acid (RNA) oligonucleotides The test strip may be made of nitrocellulose, and optionally include a sample pad and a porous wick. The sample pad may contain receptor-coated detectable nanoparticles that mix with the sample/reagent mixture when added to the pad. The test line may contain a surface immobilized receptor capable of binding either the first affinity tag or the second affinity tag but not both. The receptor can be solubilized and sprayed onto the strip and allowed to dry before use. The test strip may also include a control line located between the test line and the wicking pad. The control line may be used to verify the proper flow of the test sample fluid through the test strip. The control line may contain an immobilized receptor capable of directly binding the plurality of detectable nanoparticles regardless of the presence of ribonuclease in the test sample, and whether ribonucleic acid in the test sample is cleaved or not cleaved.

In embodiments, the control line contains immobilized, biotinylated protein and the detectable nanoparticles are coated with streptavidin. Alternatively, the receptor on the control line may be selected to bind a water-soluble linker compound selected to be capable of binding the detectable nanoparticles. In other embodiments, the control line may contain anti-digoxigenin antibodies, while the plurality of detectable nanoparticles are coated with streptavidin, and the linker compound is a DNA oligonucleotide dual-labeled with biotin and digoxigenin (DIG-DNA-TEG Biotin). The control line may function to capture the detectable nanoparticles as they flow through the test strip, creating a detectable control line independent of whether ribonuclease is present in the sample.

In embodiments, the test line of the test strip may contain an immobilized anti-FAM antibody, while the control line may contain immobilized biotinylated bovine serum albumin (BSA). The plurality of detectable nanoparticles may be coated with streptavidin and the ribonucleic acid of the reagent fluid may be tagged with biotin and FAM (fluorescein).

Coating the nanoparticles with sub-saturating amounts of RNA oligonucleotide permits a portion of the biotin-coated nanoparticles to bind to the control line. When test samples lacking RNase are added to a buffered solution containing the oligonucleotide and the nanoparticles, the oligonucleotide-coated nanoparticles bind to the test line and control line. Samples containing active RNase cause cleavage and degradation of the oligonucleotide which prevents the binding of the nanoparticles to test line. In this case, the nanoparticles only bind to the control line. Partial degradation of the oligonucleotide in samples containing low levels of ribonuclease causes a reduced test line signal and an increased control line signal. In these cases, the ratio of the test line signal to the control line signal (T:C ratio) is reduced compared to the ratio for samples lacking RNase. In one embodiment, the ratio of samples lacking RNase is greater than or equal to one, while the ratio for samples containing RNase is less than one. In this case, no comparison to a negative or positive control is needed. Our device is especially sensitive when neutravidin-coated quantum dots are used. Tests performed using samples containing 10 femtograms RNase A can produce a reduction in T:C ratio of greater than 98% compared to tests performed using samples lacking RNase, as measured using a fluorescence strip reader.

A further component of the methods and kits of the present invention is a running buffer, which is nuclease-free and configured to facilitate the flow of detectable nanoparticles and the test sample mixture along the test strip. In one example, the running buffer may contain 20 mM HEPES, pH 7.5, 150 mM NaCl, 0.4% Tween 20 detergent, and 1% acetylated BSA to promote optimal sample flow.

The present invention is useful to determine whether a test sample contains an active ribonuclease A enzyme. During the test, the test sample may be mixed with the dual-labeled ribonucleic acid and a nuclease-free aqueous running buffer near physiological pH and with a low chloride ion concentration (less than 0.15 M). All components of the invention must be free of contaminating ribonucleases to achieve maximum detection sensitivity. The running buffer can be HEPES-NaOH or Tris-HCl or other similar buffers, excluding buffers or ions known to inhibit ribonuclease activity. In one embodiment, the test sample and ribonucleic acid may be mixed in 20 mM Tris-Cl, pH 8. According to the present invention, reaction sensitivity is significantly enhanced by the addition of 0.5-5% by volume of a block copolymer surfactant, such as Pluronic F-68. The addition of Pluronic F68 copolymer to the enzyme reaction causes an enhancement of the ratio of test line signal-to-control line fluorescence emission signal (T:C ratio) and an enhanced change in the ratio caused by the presence of RNase in the test sample. The increased T:C ratio caused by the addition of Pluronic F68 is observed in assays using RNA oligonucleotides labeled with FAM (fluorescein) or DIG (digoxigenin) affinity tags.

The method for ribonuclease detection, according to the invention, may include the following steps:
a. mixing an aqueous test sample with the reagent fluid to cause them to react together, wherein the reagent fluid comprises ribonucleic acid attached to a first affinity tag and a second affinity tag in a buffered aqueous solution containing a block copolymer surfactant,
b. adding the running buffer to the mixture, wherein the running buffer is configured to facilitate flow of detectable nanoparticles along the test strip,
c. including the plurality of detectable nanoparticles as coated onto the test strip or as added to the mixture of step (a) or added to the mixture of step (b), wherein the plurality of detectable nanoparticles have their surfaces coated with a receptor capable of binding either the first affinity tag or the second affinity tag but not both, and wherein the test strip comprises a test line containing surface immobilized receptor capable of binding either the first affinity tag or the second affinity tag but not both, and
d. causing the mixture of step (g) to flow along the test strip to reach the test line,
   wherein the presence of ribonuclease in the test sample diminishes the binding of the plurality of detectable nanoparticles to the test line. In other words, detectable nanoparticles cause the formation of a detectable signal at the test line as the sample flows through the strip. However, if the sample contains ribonuclease, the ribonucleic acid is degraded, so it is incapable of simultaneously binding both receptors (the immobilized receptors at the test line position) and cannot tether the detectable nanoparticles to the test line as they flow through the test strip. Therefore, the presence of ribonuclease in a sample causes a readily detectable decrease in the test line signal. The signal intensities may be quantitated by reflectometry for colloidal particles or fluorescence excitation (using visible light) for quantum dot nanoparticles. One measure of the presence of ribonuclease may be a ratio of the test to control color intensities, or T:C ratio.

A kit for ribonuclease detection, according to the invention, may include:
a. a reagent fluid comprising ribonucleic acid attached to a first affinity tag on the first end and a second affinity tag on the second end in a buffered aqueous solution containing a block copolymer surfactant,
b. a plurality of detectable nanoparticles with their surfaces coated with a receptor capable of binding either the first affinity tag or the second affinity tag but not both,
c. a test strip having a test line containing surface immobilized receptor capable of binding either the first affinity tag or the second affinity tag but not both, and
d. a running buffer to facilitate lateral flow of detectable nanoparticles along the test strip.

The invention may be useful for the detection of a ribonuclease on hard surfaces. In one embodiment, a nuclease-free cotton swab may be immersed in an excess volume of buffered aqueous reagent solution, typically 0.2-0.4 milliliters, containing dual-labeled ribonucleic acid and block copolymer surfactant in a plastic tube. The swab may then be used to swipe the test surface and returned to the reagent solution, where it may be incubated for 1-15 minutes. The detectable nanoparticles and the running buffer may then be added to the mixture before it is transferred to the sample pad of the test strip and allowed to flow up the test strip for about 10 minutes. RNase present on the hard surface causes a strong reduction of the observed T:C ratio compared to tests performed on an identical hard surface lacking ribonuclease.

According to the invention, a method for detecting the presence of ribonuclease on a surface may include the following steps:
a. providing a nuclease-free swab,
b. providing a reagent fluid comprising ribonucleic acid attached to a first affinity tag and a second affinity tag in a buffered aqueous solution containing a block copolymer surfactant,
c. applying the reagent fluid of step (b) to the swab,
d. swiping the hard surface with the swab of step (c),
e. submerging the swab of step (d) in the remaining reagent fluid of step (a),
f. providing a plurality of detectable nanoparticles with their surfaces coated with a receptor capable of binding either the first affinity tag or the second affinity tag but not both,
g. providing a running buffer to facilitate the flow of detectable nanoparticles along the test strip,
h. applying the plurality of detectable nanoparticles and the running buffer to the reagent fluid of step (e),
i. providing a test strip having a test line containing surface immobilized receptor capable of binding either the first affinity tag or the second affinity tag but not both, and
j. causing the mixture of step (h) to flow along the test strip to reach the test line,
   wherein the presence of ribonuclease on the surface diminishes the binding of the plurality of detectable nanoparticles to the test line.

The following examples further illustrate the methods and kits of the present invention.

### Example 1

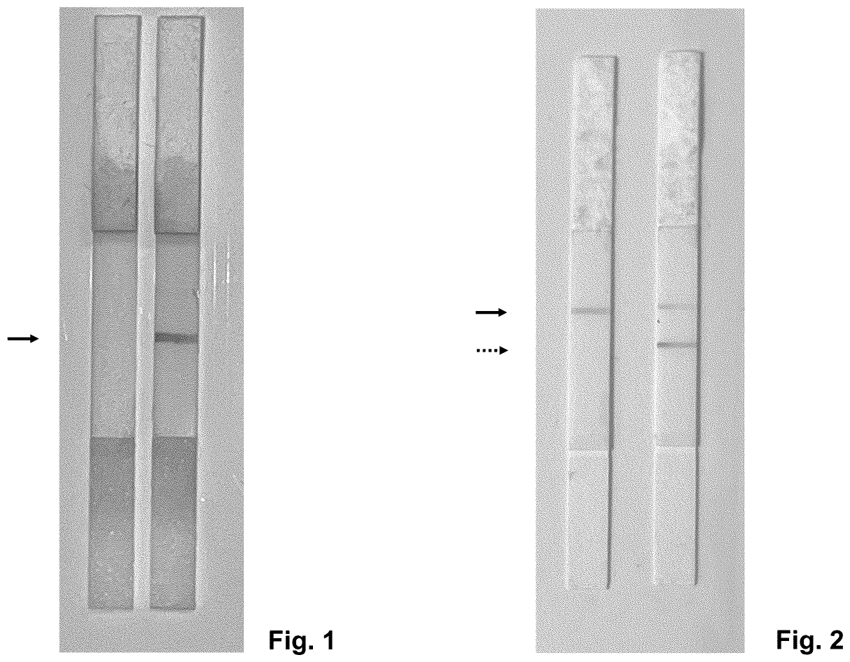

0.2 ml of 20 mM Tris-phosphate, 150 mM NaCl, and 1% Tween 20, pH 7.7, prepared using nuclease-free water, was added to two microplate wells. To one of the wells, twenty picomoles (equivalent to one microliter) of a dual-labeled ribonucleic acid was added. This ribonucleic acid, comprising the sequence 5′-AUCGUGCAACGUAGCAU-3′, contained a fluorescein (FAM) group at the 5′ end and a triethylene glycol-biotin group at the 3′ end (SEQ 3). Nitrocellulose test strips containing an anti-FAM test line and a sample pad containing streptavidin-coated gold detectable nanoparticles were immersed into the wells. The test strips were left in the wells to incubate for 10 minutes. After the incubation period, a strong visual test line signal appeared on the strip immersed in the well containing ribonucleic acid, see FIG. 1, right test strip. This signal indicates the oligonucleotide-mediated binding of gold particles to the test line. No such signal was observed for the strip immersed in the buffer lacking the oligonucleotide, see FIG. 1, left test strip.

### Example 2

0.2 ml of 20 mM HEPES-NaOH, 150 mM NaCl, and 1% Tween 20, at a pH of 7.5, prepared using nuclease-free water, was added to two microplate wells. To one of the wells, 0.5 picomole of a dual-labeled ribonucleic acid was added (SEQ 3). A dual-labeled DNA oligonucleotide (0.1 picomole) was added to both wells. This oligonucleotide, comprising the sequence 5′-GATCAACGGTACGAC-3′, contained a digoxigenin (DIG) group at the 5′ end and a triethylene glycol-biotin group at the 3′ end (SEQ 6). Nitrocellulose test strips containing an anti-FAM (test line), an anti-DIG (control line), and a sample pad containing streptavidin-coated gold nanoparticles were immersed into the wells. The test strips were left in the wells to incubate for 5 minutes. After the incubation period, a strong visual control line signal appeared on both test strips (solid arrowhead, FIG. 2) and a strong visual test line signal appeared on the strip immersed in the well containing RNA oligonucleotide (see FIG. 2, right test strip; dashed arrowhead)

### Example 3

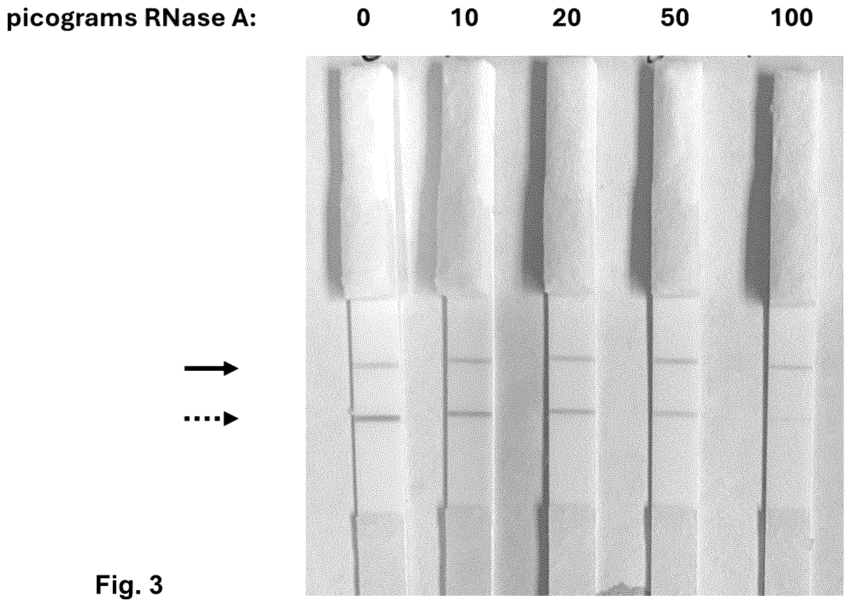

This example illustrates a dose-dependent detection of active ribonuclease using a lateral flow strip, colloidal gold nanoparticles, and a dual-labeled RNA oligonucleotide. Ribonuclease A was diluted in 75 mM HEPES-NaOH, 150 mM NaCl, pH 7.5 to a concentration of 10 picograms per microliter (pg/μl). 0-10 μl (0, 1, 2, 5, or 10) of this solution was added to wells containing 0.5 picomoles FAM/TEG-biotin labeled RNA oligonucleotide (SEG 3) and 0.1 picomole DIG/biotin labeled DNA oligonucleotide (SEQ 6) in 120 μl 33 mM Na-HEPES/1.7% Tween 20, pH 7.4 and 10 μl streptavidin-coated gold nanoparticles (10 OD). Immediately after mixing, lateral flow strips containing an anti-FAM test line and an anti-DIG control line were immersed into the wells and the samples were allowed to flow through the test strip for 5 minutes. The signal intensity of the test line (position indicated by the dashed arrowhead in FIG. 3) decreases when increasing amounts of RNase A are added to the mixture, while the control line intensity remains fairly constant (indicated by the solid arrowhead, FIG. 3).

### Example 4

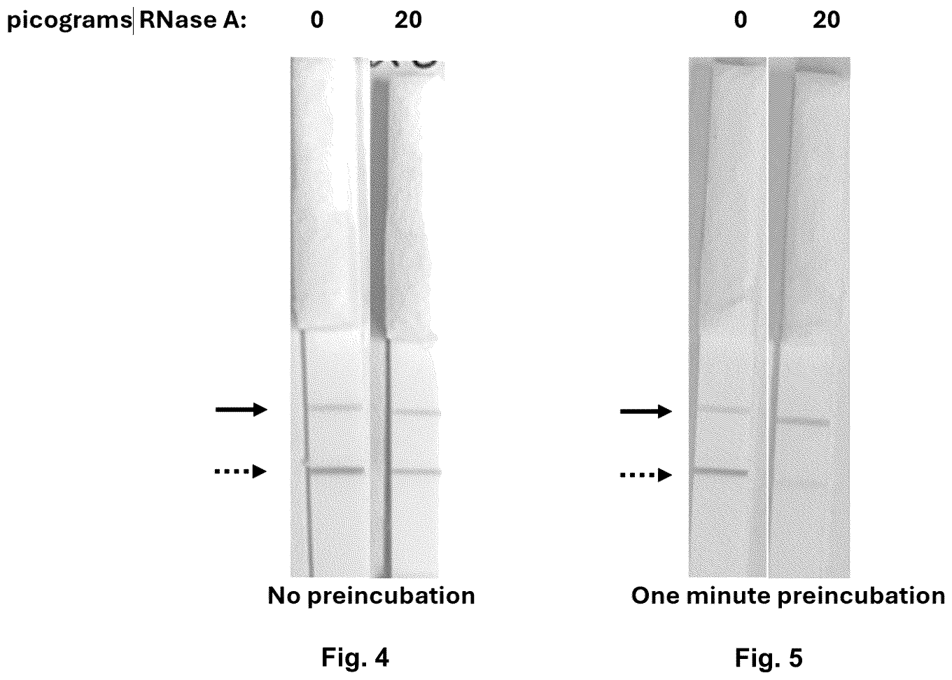

0 or 2 microliters of 10 pg/μl ribonuclease A in HEPES buffer were added to microplate wells containing 0.1 ml of 20 mM HEPES-NaOH, 1% Tween 20, pH 7.5 and dual-labeled RNA (SEQ 3) and DNA (SEQ 6) oligonucleotides (0.5 and 0.1 picomole, respectively). 0.1 ml of 20 mM HEPES-NaOH, 300 mM NaCl, 1% Tween 20, pH 7.5 was added to the wells either after a one-minute preincubation or immediately (no preincubation). Nitrocellulose strips containing an anti-FAM (test line), an anti-DIG (control line), and a sample pad containing streptavidin-coated gold nanoparticles were immersed into the wells. The strips were incubated in the wells for 10 minutes. An increased change in test line intensity caused the addition of 20 pg ribonuclease is observed when the sample is preincubated for one minute (FIG. 5) compared to the test line observed for samples treated with 20 pg RNase A without preincubation (FIG. 4).

### Example 5

5 picomoles of FAM-RNA-TEG biotin oligonucleotides of varying length, 6 to 30 nucleotides (SEQ 1, SEQ 2, SEQ 3, SEQ 4) were added to duplicate tubes containing 50 μl 20 mM HEPES, pH 7.5, 1% Tween reaction buffer containing 1 picomole DIG-DNA-TEGbiotin control oligonucleotide (SEQ 6) and 5 μl 10 OD streptavidin-coated gold. To one of the duplicate tubes, one picogram of RNase A was added. After mixing, the tubes were incubated for 15 minutes at 37° C. and then diluted with 50 μl 40 mM HEPES pH 7.5, 300 mM NaCl, 0.8% Tween 20 and 2% acetylated BSA. Test strips containing anti-FAM (test lines) and anti-DIG (control lines) were immersed in the wells for 15 minutes and then the intensities of the test and control lines of each test strip were measured using reflectometry. The ratios of the test and control lines signals (T:C) were calculated and recorded as follows.

TABLE A

| RNA substrate | RNase | Control line | Test line signal | T:C Ratio |
|---|---|---|---|---|
| 6 | − | 703632 | 596067 | 0.85 |
| 6 | + | 668127 | 516067 | 0.77 |
| 10 | − | 767981 | 454846 | 0.59 |
| 10 | + | 700886 | 161358 | 0.23 |
| 17 | − | 666676 | 342953 | 0.51 |
| 17 | + | 870266 | 67140 | 0.08 |
| 30 | − | 975986 | 634248 | 0.65 |
| 30 | + | 872990 | 11983 | 0.01 |

### Example 6A

This example illustrates how the addition of Pluronic F68 block copolymer surfactant to the enzyme reaction enhances T:C ratio and a change in T:C ratio caused by RNase, as measured using reflectometry in a test strip reader. 2 μl 300 fg/μl RNase A in 50 mM TrisCl pH 8/50% glycerol or 2 μl 50 mM TrisCl pH 8/50% glycerol buffer were added to tubes containing 100 μl 30 mM TrisCl pH 8 buffer containing 0.027 pmole/μl FAM-AUCGUGCAACGUAGCAU-TEGbiotin (SEQ3) and 0-5% Pluronic F68 surfactant. The tubes were incubated at room temperature for 10 minutes. 100 μl 2× running buffer containing 40 mM sodium phosphate pH 7.6, 300 mM NaCl, 0.8% Tween 20, 2% acetylated bovine serum albumin, and 0.06 picomole/μl DIG-GATCAACGGTACGAC-TEGbiotin (SEQ6), and 5 μl 10 O.D. streptavidin-coated colloidal gold nanoparticles were added to each tube. Nitrocellulose test strips containing an anti-FAM (test line) and an anti-DIG (control line) were immersed into the tubes and incubated for 10 minutes to allow the liquid to flow through the test strips. The intensities of the test line and control line signal were immediately measured by reflectometry using a test strip reader.

TABLE B
FAM-RNA-Biotin

| F-68 reaction | | | | | | |
|---|---|---|---|---|---|---|
| concentration | RNase A | | Control line | Test line | T:C | Ratio |
| (%) | amount | | signal | signal | Ratio | decrease |
| 0 | 0 | fg | 261473 | 785872 | 3.01 | 37% |
| 0 | 600 | fg | 359889 | 685431 | 1.90 | |
| 0.5 | 0 | fg | 218901 | 730707 | 3.34 | 84% |
| 0.5 | 600 | fg | 455075 | 236814 | 0.52 | |
| 1 | 0 | fg | 118936 | 671675 | 5.65 | 86% |
| 1 | 600 | fg | 446446 | 351831 | 0.79 | |
| 2 | 0 | fg | 199641 | 817343 | 4.09 | 62% |
| 2 | 600 | fg | 359356 | 555491 | 1.54 | |
| 5 | 0 | fg | 123873 | 672628 | 5.43 | 43% |
| 5 | 600 | fg | 211615 | 650735 | 3.07 | |

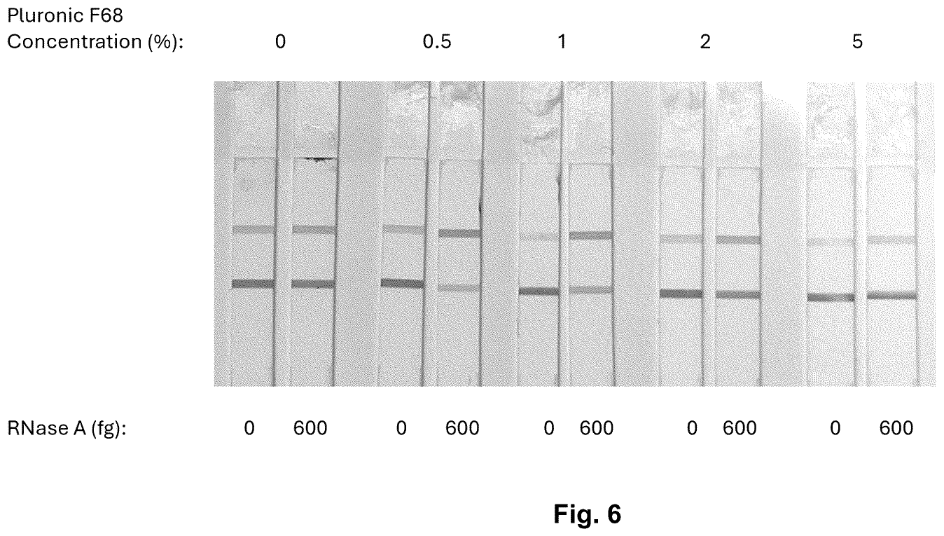

Test strips are seen in FIG. 6.

### Example 6b

5 μl 30 fg/μl RNase A in 50 mM TrisCl PH 8/50% glycerol or 2 μl 50 mM TrisCl pH 8/50% glycerol buffer were added to tubes containing 100 μl 20 mM TrisCl pH 8 buffer containing 0.032 picomole/μl DIG-AUCGUGCAACGUAGCAU-TEGbiotin (SEQ 5) and 0-5% Pluronic F68 block copolymer surfactant. The tubes were incubated at room temperature for 10 minutes. 100 μl 2× running buffer containing 40 mM sodium phosphate pH 7.6, 300 mM NaCl, 0.8% Tween 20, 2% acetylated bovine serum albumin, and 0.05 picomole/μl FAM-ATCGTGCAACGTAGCAT-TEGbiotin (SEQ 7), and 10 μl 10 O.D. streptavidin-coated colloidal gold nanoparticles were added to each tube. Nitrocellulose test strips containing an anti-DIG (test line) and an anti-FAM (control line) were immersed into the tubes and incubated for 10 minutes to allow the liquid to flow through the strips. The intensities of the test line and control line signal were immediately measured by reflectometry using a test strip reader.

TABLE C
DIG-RNA-Biotin

| F-68 reaction | | | | | | |
|---|---|---|---|---|---|---|
| concentration | RNase | | Test line | Control line | T:C | Ratio |
| (%) | amount | | signal | signal | Ratio | decrease |
| 0 | 0 | fg | 384590 | 836336 | 0.46 | 34% |
| 0 | 150 | fg | 246061 | 810303 | 0.30 | |
| 0.5 | 0 | fg | 538135 | 832379 | 0.64 | 50% |
| 0.5 | 150 | fg | 273353 | 848240 | 0.32 | |
| 1 | 0 | fg | 565413 | 791183 | 0.71 | 51% |
| 1 | 150 | fg | 306664 | 877116 | 0.35 | |
| 2 | 0 | fg | 622314 | 751095 | 0.83 | 37% |
| 2 | 150 | fg | 413667 | 800830 | 0.52 | |
| 5 | 0 | fg | 537285 | 511549 | 1.05 | 41% |
| 5 | 150 | fg | 385141 | 617963 | 0.62 | |

### Example 7

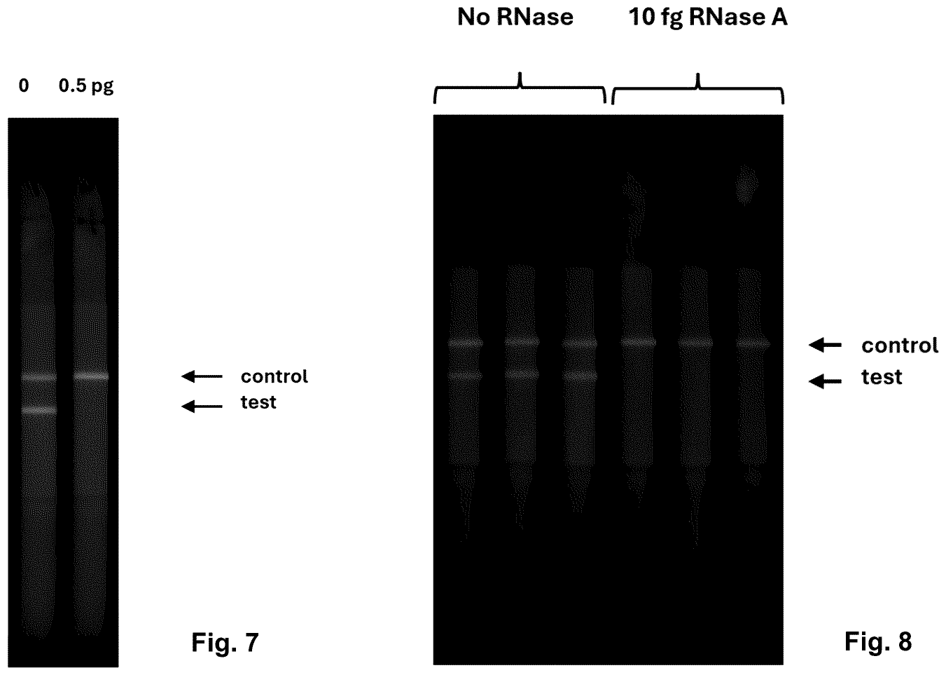

This example illustrates how quantum dots nanoparticles can be used as a plurality of detectable nanoparticles. 12.5 picomoles of FAM-RNA-TEG biotin oligonucleotides (SEQ 4), 30 nucleotides in length, were added to duplicate tubes containing 100 μl 30 mM Tris-Cl, pH 8.6, 0.75% Pluronic F-68 containing 2.5 picomole DIG-DNA-TEG biotin control oligonucleotide (SEQ 6) and 1:100-diluted neutravidin-coated Quantum dots. To one tube, 1 μl (0.5 picogram) of RNase A was added. To the other tube, 1 μl RNase storage buffer was added. After mixing, the tubes were incubated for 15 minutes at 37° C. and then diluted with 100 μl 40 mM HEPES pH 7.5, 300 mM NaCl, 0.8% Tween 20 and 2% acetylated BSA. Test strips containing anti-FAM (test lines) and anti-DIG (control lines) were immersed in the wells for 10 minutes and then the intensities of the test and control lines of each strip were measured using a fluorescence test strip reader. The ratios of the test and control line signals (T:C ratios) were calculated and recorded, see FIG. 7.

### Example 8

This is an illustration that quantum dots nanoparticles provide high test line signal reduction and detection sensitivity. 12.5 picomoles of FAM-RNA-TEG biotin oligonucleotides, 30 nucleotides in length (SEQ 4), were added to six tubes containing 100 μl 30 mM Tris-Cl, pH 8.6, 0.75% Pluronic F-68 containing 2.5 picomole DIG-DNA-TEG biotin control oligonucleotide (SEQ 6) and 0.0007 mg/ml neutravidin-coated quantum dots. To three tubes, ten femtogram of RNase A was added. To three tubes, 1 μl RNase storage buffer was added. After mixing, the tubes were incubated for 15 minutes at 37° C. and then diluted with 100 μl 40 mM HEPES pH 7.5, 300 mM NaCl, 0.8% Tween 20 and 2% acetylated BSA. Test strips containing anti-FAM (test lines) and anti-DIG (control lines) were immersed in the wells for 10 minutes and then the intensities of the test and control lines of each strip were measured using a test strip reader. The ratios of the test and control line signals (T:C) were calculated and recorded. The presence of 10 femtograms RNase A in samples caused the average T:C ratio to decrease by 98.4% (average of triplicates), from 0.7453 to 0.01199. Test strips are illustrated in FIG. 8.

| RNase | | Control | Test | Ratio | Average ratio: |
|---|---|---|---|---|---|
| 0 | pg | 2128.33 | 1341.47 | 0.630292295 | 0.74526373 |
| 0 | pg | 1836.38 | 1580.45 | 0.86063342 | |
| 0 | pg | 2142.36 | 1595.77 | 0.744865475 | |
| 0.01 | pg | 2121.01 | 18.144 | 0.008554415 | 0.011986602 |
| 0.01 | pg | 1732.58 | 47.3344 | 0.027320181 | |
| 0.01 | pg | 1718.42 | 0.146423 | 0.000852079 | |

### Example 9

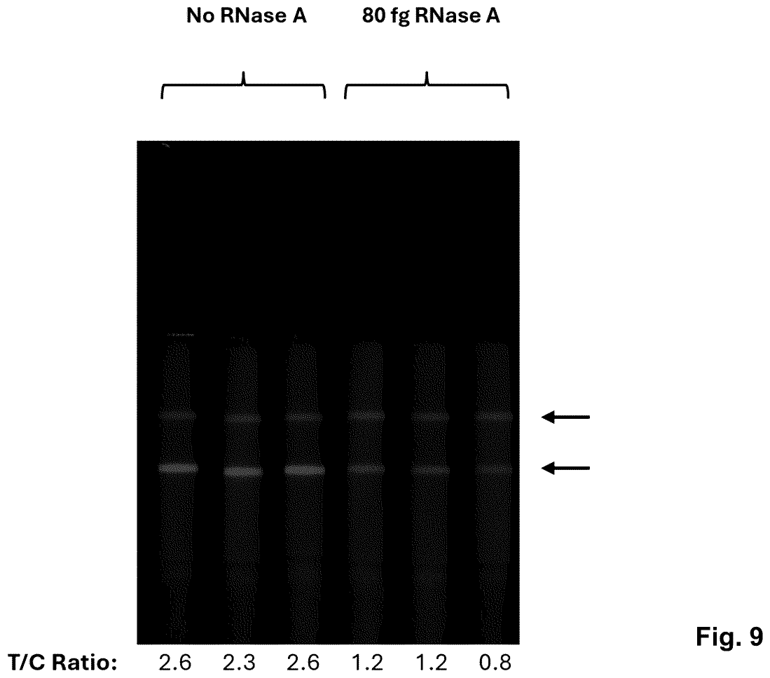

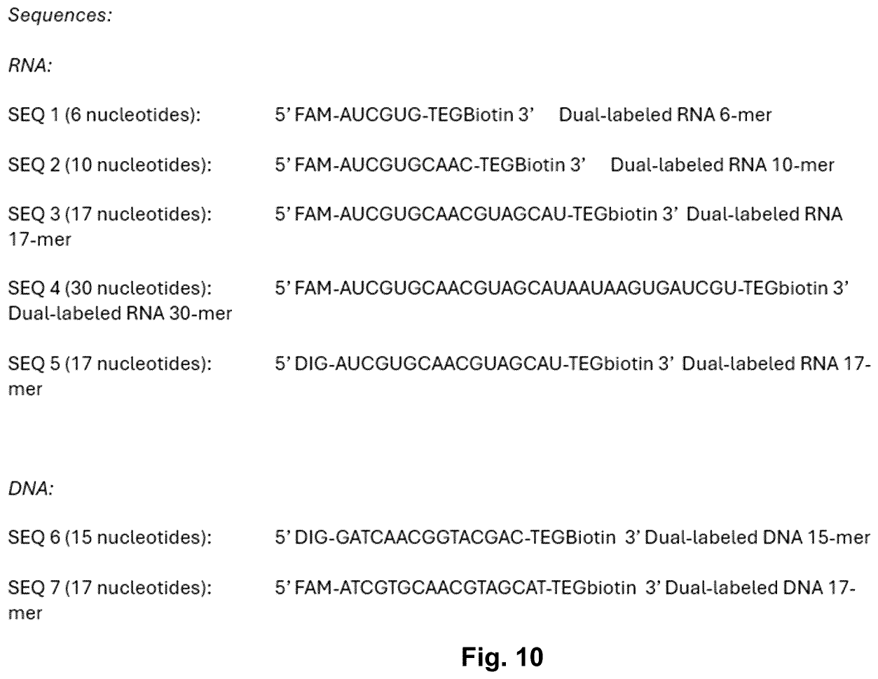

Six nuclease-free cotton swabs were each immersed in 0.35 ml 30 mM Tris-HCl PH 8.6, 0.75% Pluronic F68 containing 36 picomoles of RNA oligonucleotide, 30 nucleotides in length, labeled with biotin and fluorescein (SEQ 4) in a plastic tube. The swabs were then used to swipe 4 cm² square areas on glass plates. Three of the plates contained only nuclease-free buffer and the other three plates contained 20 femtogram/cm² ribonuclease A. After swiping, the swabs were returned to the tubes and rotated for 10 seconds. The swabs were allowed to rest in the tubes for 3 minutes and then discarded. After the tubes were incubated for 15 minutes at 37° C., 6 picomoles of DNA oligonucleotide, 15 nucleotides in length, labeled with biotin and digoxigenin (SEQ 6) were added to each tube and then a 0.1 ml aliquot of each tube was transferred to a new tube containing 0.1 ml 40 mM HEPES pH 7.5, 300 mM NaCl, 0.8% Tween 20, 2% acetylated BSA, and 0.0007 mg/ml neutravidin-coated quantum dots. Test strips containing anti-FAM (test lines) and anti-DIG (control lines) were immersed in the tubes for 10 minutes and then the intensities of the test and control lines of each strip were measured using a strip reader. The ratios of the test and control line signals (T:C ratio) were calculated and recorded. Test strips are illustrated in FIG. 9.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method for ribonuclease detection comprising the following steps:
a. mixing an aqueous test sample with the reagent fluid to cause them to react together, wherein the reagent fluid comprises ribonucleic acid attached to a first affinity tag and a second affinity tag in a buffered aqueous solution containing a block copolymer surfactant,
b. adding the running buffer to the mixture, wherein the running buffer is configured to facilitate flow of detectable nanoparticles along the test strip,
c. including the plurality of detectable nanoparticles as coated onto the test strip or as added to the mixture of step (a) or added to the mixture of step (b), wherein the plurality of detectable nanoparticles have their surfaces coated with a receptor capable of binding either the first affinity tag or the second affinity tag but not both, and wherein the test strip comprises a test line containing surface immobilized receptor capable of binding either the first affinity tag or the second affinity tag but not both, and
d. causing the mixture of step (g) to flow along the test strip to reach the test line,
wherein a presence of ribonuclease in the test sample diminishes binding of the plurality of detectable nanoparticles to the test line.

2. The method for ribonuclease detection, as in claim 1, wherein in step (c) the test strip further contains a control line distal to the test line, wherein the control line contains an immobilized receptor capable of binding the plurality of detectable nanoparticles regardless the presence of ribonuclease in the test fluid of step (g).

3. The method for ribonuclease detection, as in claim 2, wherein in step (c) the control line contains the immobilized receptor capable of directly binding the plurality of detectable nanoparticles regardless of the presence of ribonuclease in the test sample of step (g).

4. The method for ribonuclease detection, as in claim 2, wherein in step (c) the control line contains the immobilized receptor capable of binding the plurality of detectable nanoparticles regardless of the presence of ribonuclease in a test fluid of step (g), wherein the binding involves a use of a linker compound configured to bind both to the control line and to the plurality of detectable nanoparticles.

5. The method for ribonuclease detection, as in claim 1, wherein the block copolymer surfactant in step (a) is a nonionic block copolymer surfactant.

6. The method for ribonuclease detection, as in claim 5, wherein the block copolymer surfactant in step (a) comprises a central hydrophobic chain of polypropylene oxide with two hydrophilic chains of polyethylene oxide on both ends thereof.

7. The method for ribonuclease detection, as in claim 6, wherein the block copolymer surfactant in step (a) is Pluronic F68.

8. The method for ribonuclease detection, as in claim 1, wherein in step (c) the test strip is a nitrocellulose strip.

9. The method for ribonuclease detection, as in claim 1, wherein in step (b) the plurality of detectable nanoparticles are selected from a group consisting of: colloidal gold nanoparticles, quantum dots nanoparticles, lanthanide beads nanoparticles, R-phycoerythrin nanoparticles, polystyrene nanoparticles, allophycocyanin nanoparticles, and latex beads nanoparticles.

10. The method for ribonuclease detection, as in claim 1, wherein in step (a) the ribonucleic acid of the reagent fluid comprises an oligonucleotide between 5 to 30 nucleotides in length.

11. The method for ribonuclease detection, as in claim 1, wherein in step (a) at least one of the first or second affinity tags is biotin, and in step (b) the detectable nanoparticles of the plurality of detectable nanoparticles are coated with streptavidin, or avidin, or modified avidin.

12. The method for ribonuclease detection, as in claim 1, wherein in step (a) at least one of the first or second affinity tags is an antigen, and in step (c) the surface-immobilized receptor is an antibody.

13. The method for ribonuclease detection, as in claim 1, wherein in step (a) the antigen is selected from a group consisting of a fluorescein and a digoxigenin.

14. The method for ribonuclease detection, as in claim 1, wherein in step (a) a concentration of the block copolymer surfactant is between 0.1% and 5% by volume.

15. The method for ribonuclease detection, as in claim 1, wherein in step (a) the ribonucleic acid is attached to the first affinity tag on a first end and attached to the second affinity tag on a second end separated from the first end by at least 6 nucleotides.

16. The method for ribonuclease detection, as in claim 1, wherein in step (a) the ribonucleic acid is attached to the first affinity tag on a 5′ end and the second affinity tag on a 3′ end.

17. A method for detecting ribonuclease presence on a surface, comprising the following steps:
a. providing a nuclease-free swab,
b. providing a reagent fluid comprising ribonucleic acid attached to a first affinity tag and a second affinity tag in a buffered aqueous solution containing a block copolymer surfactant,
c. applying the reagent fluid of step (b) to the swab,
d. swiping the hard surface with the swab of step (c),
e. submerging the swab of step (d) in the remaining reagent fluid of step (a),
f. providing a plurality of detectable nanoparticles with their surfaces coated with a receptor capable of binding either the first affinity tag or the second affinity tag but not both,
g. providing a running buffer to facilitate a flow of detectable nanoparticles along the test strip,
h. applying the plurality of detectable nanoparticles and the running buffer to the reagent fluid of step (e),
i. providing a test strip having a test line containing surface immobilized receptor capable of binding either the first affinity tag or the second affinity tag but not both, and
j. causing the mixture of step (h) to flow along the test strip to reach the test line,
wherein ribonuclease presence on the surface diminishes a binding of the plurality of detectable nanoparticles to the test line.

18. The method detecting ribonuclease presence on a surface, as in claim 17, wherein step (e) further comprises a step of incubation of the reagent fluid with the swab submerged there for a predefined duration of time.

19. The method detecting ribonuclease presence on a surface, as in claim 18, wherein the duration of time for the incubation in step (e) is at least 1 minute.

20. The method detecting ribonuclease presence on a surface, as in claim 19, wherein the duration of time for the incubation in step (e) is at or below 15 minutes.

21. A kit for ribonuclease detection comprising:
a. a reagent fluid comprising ribonucleic acid attached to a first affinity tag on a first end and a second affinity tag on a second end in a buffered aqueous solution containing a block copolymer surfactant,
b. a plurality of detectable nanoparticles with their surfaces coated with a receptor capable of binding either the first affinity tag or the second affinity tag but not both,
c. a test strip having a test line containing surface immobilized receptor capable of binding either the first affinity tag or the second affinity tag but not both, and
d. a running buffer to facilitate a lateral flow of detectable nanoparticles along the test strip.
